(19) United States
(12) Patent Application Publication
(10) Pub. No.: US 2022/0226521 A1
(43) Pub. Date: Jul. 21, 2022

(54) Vehicle Surfaces Sterilization Equipment

(71) Applicant: Methode Electronics Malta Ltd., Birkirkara (MT)

(72) Inventor: Alexander Galea, Dingli (MT)

(21) Appl. No.: 17/571,632

(22) Filed: Jan. 10, 2022

(30) Foreign Application Priority Data
Jan. 15, 2021 (DE) 10 2021 100 832.8

(51) Int. Cl. A61L 2/10

(52) U.S. Cl. CPC A61L 2/10; A61L 2202/25; A61L 2202/14; A61L 2202/11

(57) ABSTRACT

A device for sterilizing at least one surface within an interior or at an exterior of a vehicle. The surface is susceptible to exposure to micro-organisms, bacteria, pathogens and/or viruses. The device comprising: at least one UV-C light source mounted in or in the area of the surface of the object, at least one control module for controlling the UV-C light emitted by at least one UV-C light source, and at least one user presence detection module for sensing the presence of a user using the vehicle.

### RELATED APPLICATION DATA

This application claims the benefit of German patent application ser. no. DE 10 2021 100 832.8, filed on Jan. 15, 2021, the disclosure of which is incorporated by reference.

### DESCRIPTION

The disclosure is directed to a device for sterilizing at least one surface within an interior or arranged at an exterior of an object, preferably a vehicle, to be used by a human being (user), wherein, when the object is in use, the surface being exposed to micro-organisms and/or bacteria and/or pathogens and/or viruses transmitted from the user to the surface of the object.

### PRIOR ART

The document WO 002012019800 A2 relates to a method for vehicle conditioning and provision. It particularly it relates to a method for the conditioning and provision of hired vehicles and/or used vehicles. Vehicles referred to by the state of the art are passenger cars as well as commercial vehicles. In the field of vehicle renting as well as prior to the delivery of new vehicles, there is are necessity to condition and provide vehicles. While in the field of vehicle renting, there is the need to eliminate signs of use and dirt caused by the previous hirer prior to rehiring, in the field of the delivery of new vehicles, there is the need to eliminate production residues and dirt which typically result from vehicle production. In both cases, a great number of vehicles must be conditioned within a time as short as possible to provide them for rehiring or to deliver or hand them over to a new owner.

### DEFICIENCIES OF THE STATE OF THE ART

Objects and associated surfaces or textures which are commonly used by a plurality of individuals in a public environment promote the holding of micro-organisms, bacteria, pathogens and/or viruses.

This is especially true on handles, side pockets, foot rests and/or carpets. Micro-organisms, bacteria, pathogens and/or viruses are also found on seats and arm rests or other textiles that are not easy to wipe, when used in shared or rented automobiles.

The problem further enhances itself when vehicles are shared among drivers or passengers, such as taxis and rental cars.

Other objects and associated surfaces outside the automotive industry are dispensers such as for food, drinks, medicine and other items. Levers and buttons which are used to activate the functionality of such equipment is particularly prone to being contaminated due to touching by multiple people.

### Problem to be Solved

It is the intention of the disclosure to provide a device and a corresponding system for intensively cleaning and/or sterilizing at least one surface within the interior or arranged at or on the exterior of an object, preferably a vehicle, to be used by a human being (user). The invention should remedy the deficiencies of the prior art listed above.

### Solving the Problem

The problem is solved by a device for sterilizing at least one surface within an interior or arranged at or on an exterior of an object, preferably a vehicle, to be used by a human being (user).

When the object is in use, the surface is exposed to micro-organisms and/or bacteria and/or pathogens and/or viruses transmitted from the user to the surface of the object.

The device comprises at least one UV-C light source mounted in or in the area of the surface of the object, which is exposed to the micro-organisms and/or bacteria and/or pathogens and/or viruses.

The device further comprises at least one control module, for controlling the UV-C light, emitted by at least one UV-C light source, and at least one user presence detection module, for sensing the presence of a user, using the object.

The invention is described in more detail using a car as an example. It goes without saying that the invention can also be related to other fields of application. Other fields of application may be dispensers, elevator buttons and levers or else.

By implementing the above UV-C light source one creates a convenient vehicle sterilization system within the limits of existing lighting systems in automotive engineering.

The device and the system can be implemented without affecting the styled appearance of the vehicle.

Both the device and the system can be coupled to a control module which can switch between the car's normal light and the UV-C light.

The intensity and the duration of such UV-C light exposure can be controlled in an automated or manually activated manner.

An activation sensor, a presence sensors and/or time-outs determine whether there is presence of a user in the vehicle.

As soon as there is neither the driver nor a passenger in the vehicle, thus when it is safe, the normal light of the vehicle switches to UV-C light.

Activating the light source for a predetermined period of time is sufficient to sterilize the at least one affected surface.

Since UV-C light is absorbed by normal transparent materials utilized in modern vehicles, the UV-C light source has to be placed in such a manner such that UV-C light can pass through either open spaces such as a grill type of feature or be transmitted through special grades of silica (glass).

UV-C light UV-A light and UV-B light are naturally occurring wavelengths within the UV spectrum.

The UV-C light has a shorter wavelength range. The UV-C light is absorbed by the earth's upper atmosphere.

However, the UV-C light can be artificially produced through special bulbs such as neon tubes and LED's.

Currently the LED is mostly limited to specific sterilizing equipment where sanitization is a priority. This especially applies in hospitals cleaning equipment, medical equipment and other forms of hand held sanitary equipment.

It is also known that an exposure of a few seconds up to a few minutes to UV-C light kills and/or disables potentially infectious pathogens. It goes without saying that it depends on the power and on the affected surface area, whether the UV-C light may kill or just disable infectious pathogens.

UV-C Light Source

Currently vehicles are equipped with various light sources. The scope of the light source ranging from reading lights, to footwell lights and/or ambient lighting. Normally the light source is available to aid the user in locating the actuating device.

The UV-C light source may form an integral part of a control module.

Said control module controls the energy supply to enable switching between colors. The control module may also swap between various light sources and intensity adjustments.

Simpler sources are driven by a central module which may control the same functionality mentioned above.

### Embodiments of the Invention

According to one embodiment of the invention the UV-C light source is mounted inside the hand grip of an automotive door handle. It can also be arranged inside a door pull. At any case, the UV-C light source is facing a touch pull area of said surface.

Thus, the affected areas can be sterilized directly and efficiently.

The UV-C light source can be mounted around an inside perimeter of a vehicle cup holder. The cup holder is preferably located in a central console between a driver seat and a passenger seat.

According to the invention, both the affected surfaces of the vehicle itself and other gadgets commonly used by the passengers such as cups can be clean intensively.

Another embodiment of the invention provides the UV-C light source mounted inside the vehicle reading light overhead module, located in the center front of the vehicle roof. It can also be arranged in a center mid-roof and/or on top of a rear door.

Thus, any area of the vehicle which might be exposed to micro-organisms and/or bacteria and/or pathogens and/or viruses can be sterilized.

Also, the UV-C light source may project the UV-C light on at least one seat of the driver or of any passenger. The UV-C light source may also be projected on the steering wheel.

The UV-C light source may comprise at least one LED, arranged on at least one PCBA. Other light sources can be used as well.

The invention provides the control module that switches on or off the UV-C light source. The control module controls the UV-C light duration and/or the power of exposure of the surface to the UV-C light source.

The control module communicates with the at least one user presence detection module. Thus, the UV-C light is only switched on when no driver or no passenger occupies any of the vehicle's seats.

According to the invention the user presence detection module is a door a jar and/or a switch or sensor and/or a seat belt switch.

It can also be a seat occupant sensor and/or a seat weight sensor. The presence of a driver or of a passenger can also be checked by a camera and/or by an immobilizer module. Also, a vehicle interior alarm system and/or a key location can be employed.

According to the invention the user presence detection module can be activated manually or automatically to indicate the presence of a user, using the object.

According to another embodiment of the invention the user presence detection module can be overridden by the movement of a retractable cover of a cup holder inside the vehicle. In the example of the invention the retractable cover has a switching element or a sensor monitoring the position of the cover.

Both the UV-C light source and/or the user presence detection module and/or the control module are supplied by a battery of the vehicle.

The disclosure provides a system for sterilizing at least one surface within an interior or arranged at an exterior of an object. Preferably, the object is a vehicle, to be used by a human being (user).

The system for sterilizing at least one surface includes at least one device for sterilizing. The system also comprises at least one UV-C light source mounted in, or in the area of the surface of the object.

The surface of the object, sterilized by the system is exposed to the micro-organisms and/or to bacteria and/or to pathogens and/or to viruses.

At least one control module is provided for controlling the UV-C light, emitted by at least one UV-C light source.

The system provides at least one user presence detection module for sensing the presence of a user, using the object.

### DRAWINGS

Further examples and advantageous embodiments are shown in the drawings described below.

FIG. 1 shows the working area of a driver of a motor vehicle,

FIG. 2 shows the exterior of the vehicle according to the present invention,

FIG. 3 presents an electrical circuit 28 diagram for the device and the system for sterilization of surfaces the interior of a car and

FIG. 4 shows a dashboard of a motor vehicle providing UV-C light sources to release UV-C light.

### DETAILED DESCRIPTION

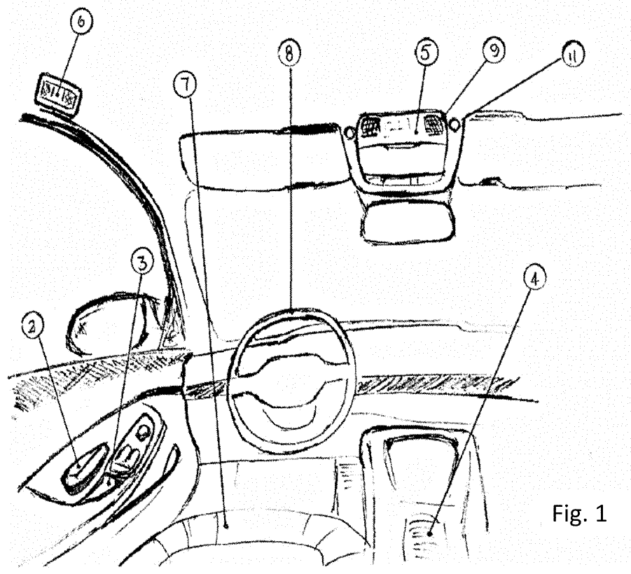

FIG. 1 shows the working area of a driver of a motor vehicle as seen from the driver's seat 7.

On the left of the driver's seat 7 one can see the door of the driver.

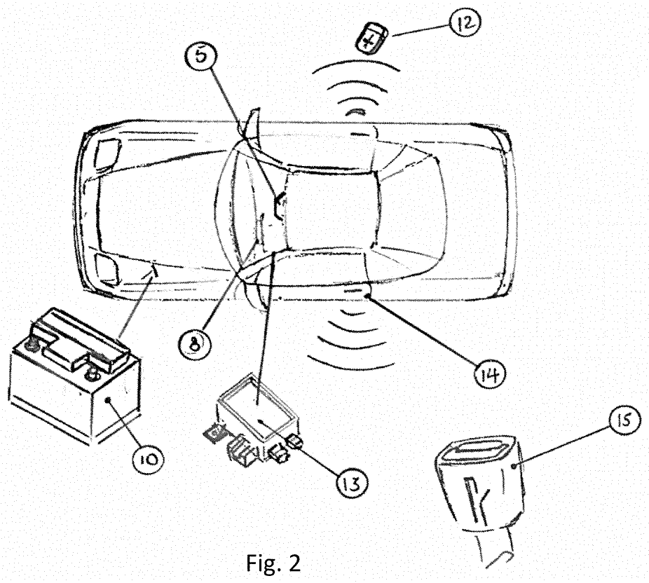

A single or multiple UV-C light source 1 (not shown) may be at least one LED. The LED is mounted inside the hand grip of an automotive door handle 2 and/or of a door pull 3. The LED is facing the touch pull surface and is controlled through a control module 16 (not shown) both of which are supplied by the vehicle battery 10 (FIG. 2).

Above the driver's door one can see a reading light overhead module 5.

In front of the driver's seat 7 FIG. 1 shows the steering wheel 8. At the right hand side of the driver's seat 7 there is the passenger seat (not shown).

Between the driver's seat 7 and the passenger seat in the vehicle there is a vehicle cup holder 4.

Another overhead module 5 is arranged above the driver's mirror on the inside of the vehicle. The overhead module 5 arranged above the driver's mirror comprises a camera 11 and a grid like geometry 9 protecting the PCBA (not shown) against touching.

FIG. 2 shows the exterior of the vehicle. Behind the front window there is the overhead module 5 above the steering wheel 8.

On either side of the vehicle the FIG. 2 shows an electronic key location 12 and the door-adjar 14 of the vehicle.

A battery 10, the presence detection module 13 and the seat belt switch 15 are arranged on the left of the vehicle.

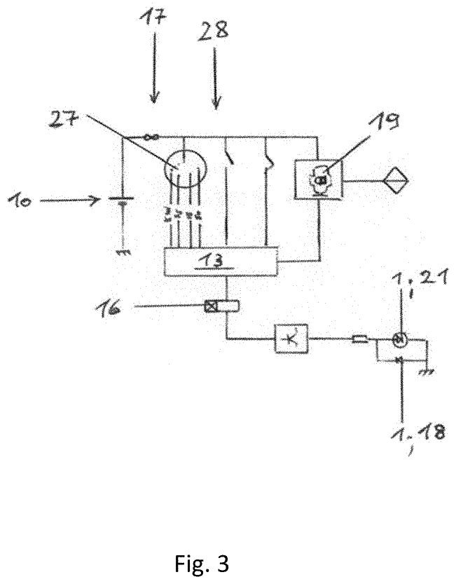

FIG. 3 shows a circuit diagram of an example of an electrical circuit 28 that can be used to operate the device 17 and the corresponding system for sterilizing at least one surface of the vehicle.

The device 17 comprises at least one UV-C light source 1 mounted in or in the area of the surface of the object, which is exposed to the micro-organisms and/or bacteria and/or pathogens and/or viruses and therefore needs to be sterilized.

The device 17 further comprises at least one control module 16, for controlling the UV-C light 21, emitted by at least one UV-C light source 1, and at least one user presence detection module 13, for sensing the presence of a user, using the vehicle.

By implementing the above UV-C light source 1; 21 one creates a convenient vehicle sterilization system within the limits of existing lighting systems in automotive engineering.

Both the device 17 and the system can be coupled to a control module 16 which can switch between the car's normal light 18 and the UV-C light 21.

The intensity and the duration of such light exposure to the UV-C light 21 can be controlled in an automated or manually activated manner.

An immobilizer module 19, a presence detection module 13 and/or time-outs determine whether there is presence of a user in the vehicle.

As soon as there is neither the driver nor a passenger in the vehicle, thus when it is safe, the normal light 18 of the vehicle switches to UV-C light 21.

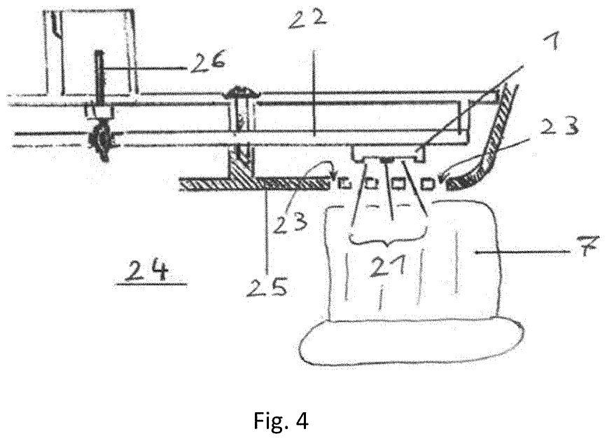

Since UV-C light 21 is absorbed by normal transparent materials utilized in modern vehicles, the UV-C light source 1 has to be placed in a manner such that UV-C light 21 can pass through either opening 23 such as a grill type of feature or be transmitted through special grades of silica (glass) (please see FIG. 4).

However, the UV-C light 21 can be artificially produced through special bulbs such as neon tubes and LED's.

Currently vehicles are equipped with various light sources 1. The scope of the light source 1 ranging from reading lights, to footwell lights and/or ambient lighting. Normally the light source 1 is available to aid the user in locating the actuating device 17.

An electronic component 27 is provided to control the electrical voltage (Volt) required for the respective light source 1 arranged within the vehicle. The electronic component 27 also controls the energy conversion per time unit (Watt) required to provide energy to the respective light source 1; 18; 21 of the vehicle.

The UV-C light source 1 may form an integral part of a control module 16.

Said control module 16 controls the energy supplies to enable switching between colors. The control module 16 may also swap between various light sources and intensity adjustments.

Simpler light sources are driven by a control module 16 which may control the same functionality mentioned above.

According to one embodiment of the invention the UV-C light source 1 is mounted inside the hand grip of an automotive door handle. It can also be arranged inside a door pull 3 (not shown). The UV-C light source 1 may also be a part of the door adjar 14. At any case, the UV-C light source 1 is facing a touch pull area of said surface (FIG. 2).

The UV-C light source 1 can be mounted around an inside perimeter of a vehicle cup holder 4 (FIG. 1). The cup holder 4 is preferably located in a central console between a driver seat 7 and a passenger seat.

Another embodiment of the invention provides the UV-C light source 1; 21 mounted inside the vehicle reading light overhead module 5, located in the center front of the vehicle roof. It can also be arranged in a center mid-roof and/or on top of a rear door (FIG. 1).

Also, the UV-C light source 1 may project the UV-C light 21 on at least one seat 7 of the driver or of any passenger. The UV-C light source 1 may also be projected on the steering wheel 8 (FIG. 1).

The UV-C light source 1 may comprise at least one LED, arranged on at least one PCBA. Other light sources can be used as well.

The invention provides the control module 16 that switches on or off the UV-C light source 1. The control module 16 controls the UV-C light 21 duration and/or the power of exposure of the surface to the UV-C light source 1.

The control module 16 communicates with the at least one user presence detection module 13. Thus, the UV-C light 21 is only switched on when no driver or no passenger occupies any of the vehicle's seats.

According to the invention the user presence detection module 13 is a door ajar 14 and/or a switch or sensor and/or a seat belt switch 15 (FIG. 1 and FIG. 2).

It can also be a seat occupant sensor and/or a seat weight sensor. The presence of a driver or of a passenger can also be checked by a camera 11 and/or by an immobilizer module 19. Also, a vehicle interior alarm system and/or a key location can be employed (FIG. 1).

According to the invention the user presence detection module 13 can be activated manually or automatically to indicate the presence of a user, using the vehicle.

According to another embodiment of the invention the user presence detection module 13 can be overridden by the movement of a retractable cover of a cup holder 4 inside the vehicle. In the example of the invention the retractable cover has a switching element or a sensor monitoring the position of the cover.

It goes without saying that the presence detection module 13 may also be overridden in any other manner.

Both the UV-C light source 1 and/or the user presence detection module 13 and/or the control module 16 are supplied by a battery 10 of the vehicle.

The electrical circuit 28 diagram comprises further electronic elements such as a resistance and/or an electric fuse.

The electric energy to run the device 17 and/or the system is provided by an energy supply such as a battery 10. Obviously, the energy may also be provided by any other means.

FIG. 4 shows a dashboard 25 of a motor vehicle. Behind the dashboard 25 of the vehicle there is at least one UV-C light source 1 provided to release UV-C light 21.

The UV-C light sources 1 emits the UV-C light 21 and directs the UV-C light 21 towards the driver's seat 7 or the passenger seat, whichever seat is located opposite to the UV-C light source 1 in the interior 24 of the vehicle.

Alternatively, the UV-C light sources 1 arranged behind the dashboard 25 of the vehicle may also direct the UV-C light 21 towards a door ajar 14 (not shown) in the FIG. 4 or any other object or component in the interior 24 of the vehicle which needs to be sterilized by means of UV-C light when the vehicle is used by varying passenger.

Between the dashboard 25 and the front-end of the vehicle (not shown) the at least one UV-C light source 1 is mounted on a carrier 22.

The UV-C light source 1 may reach through at least one opening 23 in the dash-board 25 to emit the UV-C light 21 facing away from the carrier 22 holding the UV-C light source 1 into the interior 24 of the vehicle (not shown).

Next to the at least one UV-C light source 1 a steering column 26 is arranged, supporting the steering wheel 8 of the motor vehicle.

### REFERENCE LIST

1 UV-C light source
2 Automotive door handle
3 Door pull
4 Cup holder
5 Overhead module
6 free
7 Driver seat
8 Steering wheel
9 Geometry
10 Battery
11 Camera
12 Key location
13 User presence detection module
14 Door adjar
15 Seat belt switch
16 Control module
17 Device
18 Normal light
19 Immobilizer module
20 free
21 UV-C light sources
22 Carrier
23 Opening
24 Interior of the vehicle
25 Dashboard
26 Steering column
27 Electronic component
28 Electrical circuit

1. A device for sterilizing at least one surface of a vehicle, the surface being at least one of an interior of the vehicle or arranged at an exterior of the vehicle, the surface being susceptible to exposure to at least one of micro-organisms, bacteria, pathogens, and viruses, the device comprising:
at least one UV-C light source mounted in or adjacent to the surface, which is exposed to the micro-organisms and/or bacteria and/or pathogens and/or viruses, and
at least one control module adapted and configured for controlling UV-C light emitted by at least one UV-C light source, and
at least one user presence detection module for sensing the presence of a user using the vehicle.

2. The device of claim 1, wherein the UV-C light source is mounted in at least one location of the vehicle, the at least one location comprising:
(i) a hand grip of a vehicle door handle and facing a touch pull area of the hand grip;
(ii) inside a door pull and is facing a touch pull area of the door pull;
(iii) an inside perimeter of a vehicle cup holder;
(iv) a central console between a driver seat and a passenger seat;
(v) inside a vehicle reading light overhead module;
(vi) a center front of the vehicle roof;
(vii) a center middle of the vehicle roof;
(viii) a top of a rear door of the vehicle;
(ix) a seat of the vehicle; and
(x) a steering wheel of the vehicle.

3. The device of claim 1, wherein the UV-C light source comprises at least one LED, arranged on at least one printed circuit board.

4. The device of claim 1, wherein the control module is adapted and configured to perform at least one of the following: (i) energize the UV-C light source, (ii) deenergize the UV-C light source; (iii) controls the UV-C light duration; and (iv) control a power of exposure of the surface to the UV-C light source.

5. The device of claim 1, wherein the control module communicates with the at least one user presence detection module.

6. The device of claim 1, wherein the user presence detection module is at least one of: (i) a door ajar sensor; (ii) a vehicle function operational switch or sensor, (iii) a seat belt switch; (iv) a seat occupant sensor; (v) a seat weight sensor; (vi) a camera; (vii) an immobilizer module; (viii) a vehicle interior alarm system; (ix) a key location sensor.

7. The device of claim 1, wherein the user presence detection module can be activated manually or automatically to indicate the presence of a user using the vehicle.

8. The device of claim 1, wherein the user presence detection module can be overridden by a movement of a retractable cover of a cup holder, with the retractable cover having a switching element or a sensor monitoring the position of the cover.

9. The device of claim 1, wherein a battery of the vehicle supplies power to at least one of the UV-C light source, the user presence detection module, and the control module.
